(19) United States
(12) Patent Application Publication
(10) Pub. No.: US 2023/0107303 A1
(43) Pub. Date: Apr. 6, 2023

(54) DENTAL MIRROR CLEANING SYSTEM

(71) Applicant: Ozair Banday, Boston, MA (US)

(72) Inventor: Ozair Banday, Boston, MA (US)

(21) Appl. No.: 17/664,141

(22) Filed: May 19, 2022

Related U.S. Application Data
(60) Provisional application No. 63/262,048, filed on Oct. 4, 2021.

(51) Int. Cl. A61B 1/253; A61B 1/12

(52) U.S. Cl. CPC A61B 1/253; A61B 1/126

(57) ABSTRACT

An optical device handle with an integral cleaning system includes a handle body with a mounting adapter and an actuator. The handle body has a longitudinally formed fluid conduit including an inlet and an outlet. An optical device is mounted to the handle. The actuator activates a flow of working fluid through the fluid conduit towards the optical device. The device clears debris, condensate, and other matter in situ from a dental mirror with a pressurized or free-flowing fluid to maintain the effectiveness of the reflective surface. The fluid is controlled by the user to clear the mirror without relying on a separate piece of equipment or a second operator. The invention is broadly applicable to dental, medical, and other applications.

### CROSS-REFERENCE TO RELATED APPLICATION

This application claims the benefit of priority of U.S. provisional application No. 63/262,048, filed Oct. 4, 2021, the contents of which are herein incorporated by reference.

### BACKGROUND OF THE INVENTION

The present invention relates to dental tools and, more particularly, to an integrated dental mirror with actuated surface cleaning using fluid jets.

Almost all dental procedures require the use of specialized devices to assist in both access to and visibility of various difficult-to-reach areas of the oral cavity. Of utmost importance to the dental care professional is visibility to the area of work. To facilitate this, specialized mirrors are used. These mirrors are small and mounted to an angled handle to allow for their use in various orientations. While these mirrors are critical to provide visibility, they must be used with other tools and devices within the oral cavity.

The dentist's vision is often obscured due to condensate or debris on the mirror surface. In such cases, work must be paused or stopped to allow for cleaning of the mirror until visibility is restored. This cleaning process has many downfalls, however. The cleaning process usually requires a blast of air and/or water from a secondary device inserted into the oral cavity. This poses a problem in cases where many other tools are being used at the same time, or for patients with small oral cavities in which space limitations may impede the use of an additional tool. The blast of fluid may require the mirror to be physically cleaned with a sterile gauze by a second operator/assistant. Manually cleaning the mirror often requires special absorbent material that adds cost. Moreover, the cleaning process is often performed by a second person. The dental professional performing the work does not control the mirror cleaning. Therefore, the dentist must demand cleaning by the support operator. The constant communication required between two or more people is inefficient.

No current dental tool allows for single operator use. Prior art systems are too complicated to produce and manufacture on a large scale.

As can be seen, there is a need for a system enabling the dentist to maintain agency over their visibility while performing procedures with the smallest form factor and minimum of devices.

### SUMMARY OF THE INVENTION

In one aspect of the present invention, an optical device handle having an integral cleaning system comprises a handle body having a mounting adapter at a first end and a fluid conduit formed longitudinally through the handle body, the fluid conduit including a fluid inlet and a fluid outlet; and an actuator operative to activate a flow of working fluid through the fluid conduit.

In another aspect of the present invention, an indirect visualization tool comprises a handle having a fluid conduit formed longitudinally therethrough, the fluid conduit including a fluid inlet and a fluid outlet; an optical device mounted to the handle; and an actuator operative to actuate flow of a working fluid through the fluid conduit; wherein the fluid outlet is operative to project the flow of the working fluid towards the optical device.

In another aspect of the present invention, a device comprises a cylindrical body having an integral fluid conduit; attachment means operative to couple the cylindrical body with an optical tool handle; and an actuator operative to actuate a flow of working fluid through the integral fluid conduit.

These and other features, aspects and advantages of the present invention will become better understood with reference to the following drawings, description, and claims.

### BRIEF DESCRIPTION OF THE DRAWINGS

FIG. 1 is a perspective view of a mirror and handle assembly according to an embodiment of the present invention;

FIG. 2 is another perspective view thereof;

FIG. 3 is a sectional view thereof, taken along line 3-3 in FIG. 2;

FIG. 4 is a perspective view thereof, shown in use;

FIG. 5 is a perspective view of a mirror and handle assembly according to another embodiment of the present invention;

FIG. 6 is a sectional view thereof;

FIG. 7 is a side elevation view thereof;

FIG. 8 is a detail view thereof, showing a nozzle according to an embodiment of the present invention; and

FIG. 9 is a detail view thereof, showing a nozzle according to another embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a cleaning system for clearing debris, condensate, and other matter from an optical tool in situ with an integrated fluid supply.

The present invention is not limited to use with a dental mirror. Any device that is cleaned with a fluid jet may utilize the inventive cleaning system. Such a device may be used with optics or video in fields including and external to medicine, such as other areas of medicine that rely on indirect vision, optical work in the rough-in industry, and advertisements. For example, the present invention may provide manually actuated or automated fluid flow to remove debris from a camera or optical lens. For brevity and simplicity, the present disclosure primarily refers to a dental mirror as the device or tool provided with an integrated cleaning system.

The device comprises a handle with an integrated fluid supply. It may be used with a pressurized or free-flowing fluid to clear away solid or liquid debris from the surface of the mirror to maintain the effectiveness of the reflective surface in use.

Any debris that might obstruct the view and impede the reflective ability of a dental mirror, such as liquid droplets, dust, pastes, tooth or bone debris, tissue, or other such obstructive material may be removed with the inventive system. Air or fluid flow is directed via jet towards a mirror surface to clear the mirror. The flow enables debris and condensate to be easily removed for greater visibility and efficiency while working.

In some embodiments, the fluid momentum, in interacting with debris, propels it off the surface of the mirror and away to restore visibility. Fluid debris may be removed from the surface by means of evaporation or vaporization by the fluid, thus restoring visibility. In other embodiments, a fluid having a viscosity effective to drag the debris away through viscous or friction forces is used.

In some embodiments, the handle and mirror assembly may be a single integrated device.

In other embodiments, the mirror assembly and handle may be separate, releasably coupled components, which may facilitate, for example, cleaning and sterilization, personalization, and patient customizability. The handle may include attachment points or attachment means for a mirror assembly, such as a mounting adapter. For example, a mirror assembly may fit into a mirror adaptor/interface at one end of the handle

In some embodiments, the device may be attached in parallel to an existing tool. The handle body may be piggybacked to a second tool handle via attachment means which may include, but are not limited to, clips, adhesive, and screws.

Some embodiments of the device may include one or more outlet conduits and one or more tool adapters/interfaces depending on the application.

Flow may be controlled on the handle or external to the handle via an actuator controlled by the user. In some embodiments, the device may incorporate actuation wherein the user manually depresses a button or other such actuator on the handle body to enable and/or disable the flow or to control the flow rate or pressure of the fluid through the conduit. For example, a button placed on the handle may be used to start a stream of air when depressed and to stop when released. Alternatively, or in addition, the actuator may be external, operated remotely, such as a foot pedal, to enable and/or disable the flow or to control the flow rate or pressure of the fluid through the conduit. In some cases, the device may be configured to have a continuous flow by default. The actuator may be either passive or active and may have mechanical, electronic, or feedback control.

In some embodiments, a pressure sensor may actuate the compressor component when it senses a drop in pressure.

In some embodiments, the actuator may be binary, as with a solenoid or other binary actuation system, where the fluid outlet is either on or off. In other embodiments, the actuator may enable some adjustment or pressure sensitivity where the fluid flow rate or pressure may be modified by the actuator. In this case, the actuator may achieve multiple fluid flow states or settings with either discrete or continuous output settings.

In some embodiments, inlet and outlet conduits, a reservoir, and the actuator are integrated within the handle. In some embodiments, the conduits, reservoir, and actuator may be affixed to the outside of the handle or otherwise integrated to achieve an on-demand, pressurized or otherwise propelled fluid at the outlet conduit. The reservoir may be centrally located within the handle or at one end. For example, an air supply provided through the inlet conduit may travel through the handle to a reservoir towards the outlet conduit.

In some embodiments, the handle may exclude a reservoir, in which case one or more inlet conduits may be directly connected to one or more outlet conduits. Alternatively, the handle may have a single conduit serving as both inlet and outlet. The single conduit may have an enlarged region serving as a reservoir.

The device uses fluid jets via either a conduit within the handle body or attached thereto. Embodiments employing a plurality of jets are contemplated. These jets may be on the mirror side of the handle or on another face to properly direct the fluid jet or jets to the tool to be cleaned. The flow may be adjusted to the mirror via the nozzles or via a shank with at least one adjustment joint. Fluid flow may be optimized via an internal reservoir, heating or cooling elements, and nozzles at the device outlet. The shape of the fluid jet is not particularly limited and may be altered by varying the pressure via either a multi-setting or continuous actuator, by variation of the inlet and outlet conduit lengths and diameters, or by other means. The cross-section shape of the jet may also be changed or optimized by altering the shape or geometry of the outlet conduit or with the introduction of a nozzle. The jet shape may be optimized to achieve a specific goal or to optimize the cleaning process depending on the specific orientation and geometry of the mirror assembly. The jet spread, being dependent on the fluid exit velocity, may be varied by changing the pressure or fluid flow rate.

In some embodiments, a nozzle or other flow modifying device used with the outlet conduit may be housed inside the handle at the outlet conduit exit. Alternatively, the nozzle may be installed external to the conduit exit. Such a nozzle or flow modifying device may be integrated, as part of the handle or conduit, or separate as an outlet attachment to facilitate purposes including, but not limited to, customization of use, fluid flow pattern, cleaning, and sterilization. The outlet conduit may include a specific nozzle to achieve a predetermined fluid jet shaped for a particular use. Such nozzles may be, for example, round, crimped, or square/rectangular.

The cleaning system utilizes a working fluid. The working fluid may originate from a local or remote source such as a pressure tank, compressor, fan, turbine, or other device effective to increase the pressure or velocity of the working fluid. The pressure may be adjusted to achieve a predetermined outlet velocity

In some embodiments, the compressor may be housed within the handle. As the fluid is compressed within the handle, an external source of pressurized fluid is unnecessary.

In some embodiments, the working fluid is ambient air and the compressor component intakes air directly from the environment. As such, the compressor component may act as a fluid flow transformer.

In some embodiments, the device may be integrated into a compressed air supply provided to a dental chair or workstation to operate other devices. For example, a coupling mechanism or supply adapter may couple a dental chair air pipe with the mirror handle. Once connected, the mirror handle has a channel for air to stream onto the mirror.

The device may be powered via an external power supply, such as integration with a dental chair or workstation, or internally, such as a battery.

In some embodiments, a heat transfer element may be present in the reservoir to change the temperature of the working fluid. A heating coil and controller may be used in the reservoir to adjust the fluid temperature and/or improve condensate clearing. The heat transfer element may alternatively be in another component of the device. The heat transfer element may be controlled by a controller which may be internally integrated or may be external to the device. For example, the controller may be contained within the conduit or integrated with a fluid supply. The controller may include, for example, a microchip or circuitry with a temperature sensor to measure the fluid temperature such that the controller increases voltage to a heating element when the measured fluid temperature falls below a predetermined range and decreases the voltage when the temperature is above the predetermined range.

In some embodiments, a pressure sensor may be present within the reservoir to actuate a compressor via a controller when the sensed fluid pressure falls below a predetermined range.

The primary user may use inventive cleaning system by holding the handle while using the mirror tool. When condensate builds up on the mirror surface, the user actuates an actuator, either on the handle or by other means, such as a foot pedal, to initiate a fluid jet aimed at the mirror surface. The fluid jet quickly clears away the condensate and restores visibility without relying on a separate piece of equipment or a second operator. The inventive device enables the user to work more efficiently and provides more reliable visibility.

The materials and methods of manufacturing the inventive device are not particularly limited. The mirror handle may be fabricated of plastic or metal, such as via three-dimensional (3D) printing or by machining a conduit into a piece of metal bar stock by drilling a hole of a predetermined diameter from one end of the piece to the opposite end. A larger hole may be made in the same end to a predetermined depth. Attachment points may be manufactured by stamping sheet metal to a predetermined shape, custom machining from metal stock, or injection molding (if using plastic). A second piece of bar stock with a conduit hole, or tubing with a predetermined diameter, may be used as a second half of the handle/body. Screwing the two halves together, or otherwise affixing them, creates the whole handle/body with conduit and integrated reservoir. A heating/cooling element may be installed through radial holes drilled into the reservoir. Nozzles may be made via bar stock or tubing of a predetermined diameter. The outside surface of the nozzle inlet may be threaded using a die such that the nozzle threads mate to threads on the handle or body, or the nozzle may be machined into the outlet of the handle/body. A preassembled actuator may be installed by machining a slot or mating plate and affixing it directly to the handle/body.

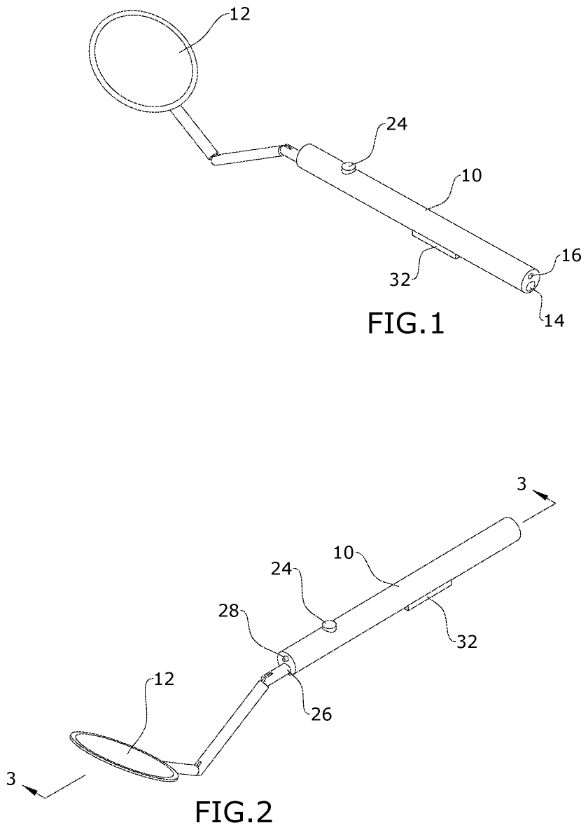

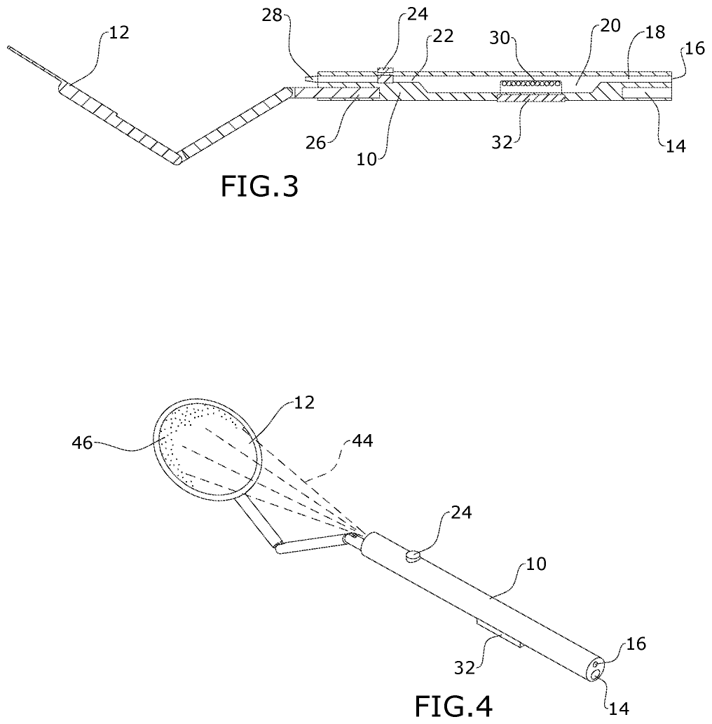

Referring to FIGS. 1 through 9, FIGS. 1 through 4 illustrate a dental mirror 12 having a shank with adjustment joints extending from a cylindrical handle 10 with an integrated cleaning system. The mirror 12 is coupled to the handle 10 by way of a mounting adapter/tool interface 26 within the handle 10. A working fluid may be delivered to a nozzle 28 in the handle 10 via a dental chair adapter 14 and an air in port 16 when a user actuates an actuator 24 protruding from an outer surface of the handle 10. Characteristics of the working fluid, such as the temperature and/or pressure, may be adjusted by a controller 32 protruding from the outer surface of the handle 10 at a position substantially antipodal from the actuator 24. As shown in FIG. 3, the working fluid (e.g., air) enters via the air in port 16, passes through an inlet conduit 18 into a reservoir 20 containing a heating coil 30, through the outlet conduit 22, and exits via the nozzle 28 which delivers a jet 44 of the working fluid onto the mirror 12 surface to remove condensation 46 or debris; see FIG. 4.

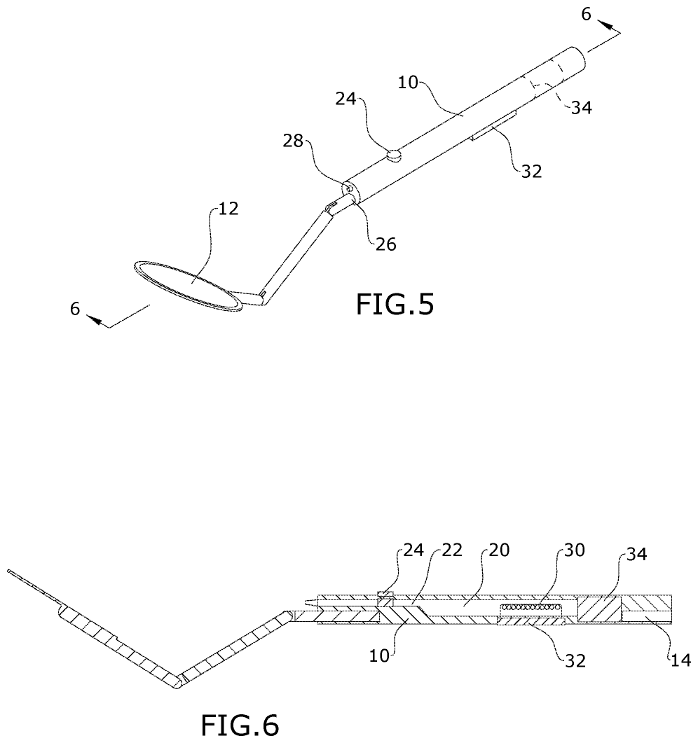

An alternate embodiment of the handle 10 is shown in FIGS. 5 and 6. In this embodiment, a compressor 34 is housed within the handle.

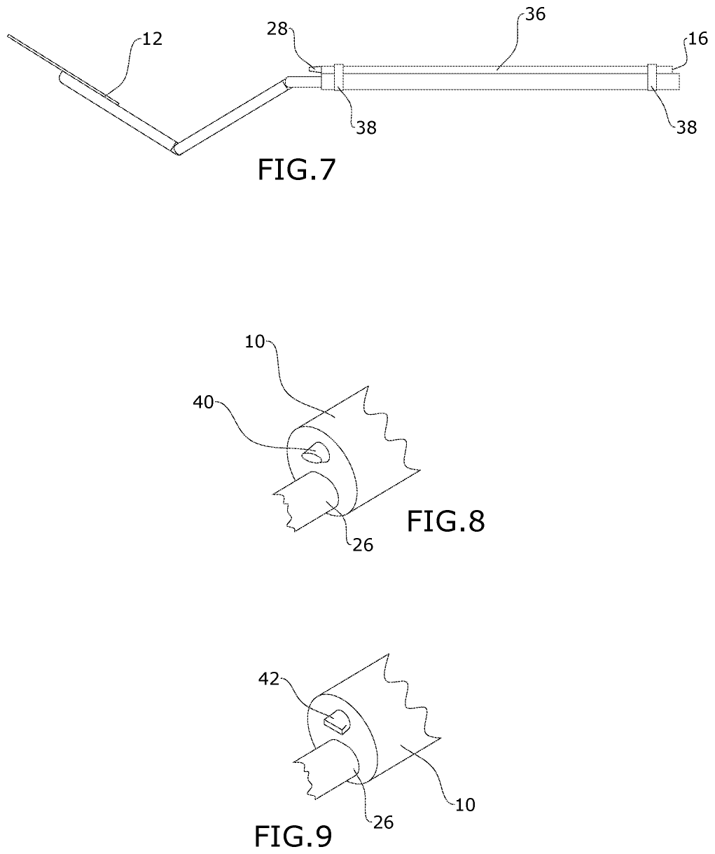

FIG. 7 illustrates an embodiment having a piggyback handle 36 which couples with a prior art mirror handle by way of handle attachments 38. Working fluid enters the air in port 16 and exits through the nozzle 28.

The jet 44 may be altered by use of a differently shaped nozzle 40, 42 such as those illustrated in FIGS. 8 and 9.

It should be understood, of course, that the foregoing relates to exemplary embodiments of the invention and that modifications may be made without departing from the spirit and scope of the invention as set forth in the following claims.

What is claimed is:

1. An optical device handle having an integral cleaning system, comprising:
a handle body having a mounting adapter at a first end and a fluid conduit formed longitudinally through the handle body, the fluid conduit including a fluid inlet and a fluid outlet; and
an actuator operative to activate a flow of working fluid through the fluid conduit.

2. The optical device handle of claim 1, further comprising a dental mirror attachment comprising a jointed shank mated with the mounting adapter.

3. The optical device handle of claim 1, further comprising a compressor housed within the handle body.

4. The optical device handle of claim 1, wherein the actuator is housed within the handle body.

5. The optical device handle of claim 1, further comprising a nozzle coupled with the fluid outlet.

6. The optical device handle of claim 1, further comprising an inlet adapter operative to couple the fluid inlet to a fluid source.

7. The optical device handle of claim 6, wherein the inlet adapter further comprises a power inlet operative to power the optical device handle.

8. The optical device handle of claim 1, further comprising a reservoir formed in the handle body and fluidly communicating with the fluid conduit.

9. The optical device handle of claim 8, further comprising a heat transfer element housed within the reservoir and a controller housed within the handle body, electrically communicating with the heat transfer element.

10. An indirect visualization tool, comprising:
a handle having a fluid conduit formed longitudinally therethrough, the fluid conduit including a fluid inlet and a fluid outlet;
an optical device mounted to the handle; and
an actuator operative to actuate flow of a working fluid through the fluid conduit;
wherein the fluid outlet is operative to project the flow of the working fluid towards the optical device.

11. The indirect visualization tool of claim 10, wherein the actuator is mounted on the handle.

12. The indirect visualization tool of claim 10, wherein the fluid conduit further comprises a nozzle configured to shape a jet of the working fluid and operative to direct the jet of working fluid onto the optical device.

13. The indirect visualization tool of claim 10, further comprising a compressor housed within the handle, fluidly communicating with the fluid conduit, and operative to propel the working fluid.

14. The indirect visualization tool of claim 13, further comprising an inlet adapter including a fluid intake interface fluidly communicating with the fluid inlet and an electrical intake interface electrically communicating with the actuator and the compressor.

15. The indirect visualization tool of claim 10, wherein the fluid conduit is operative to exhaust fluid continuously onto the optical device in an actuated condition.

16. The indirect visualization tool of claim 10, wherein the fluid conduit comprises a reservoir.

17. The indirect visualization tool of claim 16, wherein a heat transfer element is housed within the reservoir.

18. A device comprising:
a cylindrical body having an integral fluid conduit;
attachment means operative to couple the cylindrical body with an optical tool handle; and
an actuator operative to actuate a flow of working fluid through the integral fluid conduit.

19. The device of claim 18, further comprising at least one nozzle fluidly communicating with the integral fluid conduit.

20. The device of claim 18, further comprising a fluid source interface fluidly communicating with the integral fluid conduit.
